(19) United States
(12) Patent Application Publication
(10) Pub. No.: US 2024/0375061 A1
(43) Pub. Date: Nov. 14, 2024

(54) NANOPOROUS MEMBRANE

(71) Applicant: OSTIA TECHNOLOGIES LIMITED, Salford (GB)

(72) Inventor: Ka Wai WONG, Salford (GB)

(21) Appl. No.: 18/316,968

(22) Filed: May 12, 2023

(51) Int. Cl. B01D 69/12; B01D 71/02

(52) U.S. Cl. CPC B01D 69/1213; B01D 71/0213; B01D 71/024; A61M 1/16; B01D 2257/10; B01D 2323/28; B01D 2325/021; B01D 2325/02832; B01D 2325/02833; B01D 2325/02834; B01D 2325/26; C02F 1/4691

(57) ABSTRACT

A nanoporous membrane fabrication method is formed using an array of sacrificial nanopillars of removable materials are printed onto a substrate. After serial deposition of overlayers of even dissimilar nature, the sacrificial nanostructures are dissolved, leaving nanoporous membrane with nanopores, channels and cavities of nanoscale dimension and geometry designed, enabling untapped and unique functions in different technological areas such as biological artificial organs, nanoelectronics, bioelectronics, molecular sensors, and biomedical applications.

### BACKGROUND

### Technical Field

The disclosed technology pertains to nanoporous membrane fabrication using sacrificial nano-pillars of removable materials.

### Background Art

Nanoporous membranes are becoming a critical component in many different fields, from more traditional electronics, biomedical and chemical applications such as electrochemical storage devices, desalination, and chemical separation, to more frontier areas such as bioartificial organs, tissue engineering and nanophotonics.

A passive nanoporous membrane can separate or filter nano-sized entities from a mixture regardless of their material nature (organic, inorganic, or biological). Such nanoporous membranes are already an important element in many applications; however, if the nanoporous membrane possesses components through which external stimulus or excitation can be applied, it becomes an active one offering tunable and controllable characteristics and enabling more sophisticated applications.

Nanoporous membranes are conventionally prepared by several techniques. Micro machining (e.g., CNC machining, turning, milling, micro-electrical discharge machining) is a traditional machining technology but can only reach a feature dimension and resolution of μm or sub-μm level. Also, materials suitable for such machining are limited to metals and ceramics.

Chemical etching methods such as ion-track etching, anodizing, and more recently controlled break down etching, can only generate nanopores with a relatively wide size distribution. Further, the nanopores generated are randomly distributed within a membrane. Active components like an electrode or on-chip circuit cannot be pre-designed and finally fabricated. On the other hand, many of the chemically etched nanoporous membranes are polymer-based. As heterogeneous elements, for instance, metal electrodes can hardly be included. For the purposes of the present disclosure, heterogeneous means that the material is not the same physically throughout its bulk. Therefore, they usually serve as a passive membrane. Extra physical input such as electrical potential, heat, and light cannot be easily incorporated to generate additional and desirable interaction with surroundings, and thus to broaden its functionality and application scope.

General lithographic and nanoscale fabrication techniques (e.g., atomic layer deposition, scanning force lithography, nanoimprint, dip-pen nanolithography) can create a nanoscale pattern and nanocavity, which are alternative ways to prepare nanoporous membranes, but they are generally applicable on the fabrication of a thin film structure. High-aspect ratio nanoscale features, for example vials, channels, reservoirs, and compartments, cannot be easily prepared. Moreover, the corresponding processes are usually complicated, costly, labour intensive, and in some cases very difficult to scale up.

Owing to the rapid advancement of nanoscience and nanotechnology in the past decade, nanoporous membranes can also be formed using nanoparticles, nanotubes, nanowires, and other nanostructures. These methods of forming a nanoporous membrane cannot be readily used. On the other hand, as the equipment and nanomaterials involved are usually uniquely designed and manufactured at the laboratory level, they are difficult to reproduce in mass scale, rendering production extremely challenging if not impossible.

There are globally 2.6 million patients that receive renal replacement therapy with either dialysis or a kidney transplant, which is expected to double by 2030 (International Society of Nephrology, Global Kidney Health Atlas, https://www.theisn.org/initiatives/global-kidney-health-atlas, updated 2019; Accessed Feb. 4, 2022). Owing to this demand, a bioartificial kidney is one of the most sought-after technologies in medical science, since the invention of the first dialysis machine in 1943 by Willem Kolff. In particular, a wearable and implantable bio-artificial kidney has the potential to provide continuous dialysis throughout the day (Hamid Rabb, Kyungho Lee, and Chirag R. Parikh, Beyond kidney dialysis and transplantation: what's on the horizon?, The Journal of Clinical Investigation, 2022 13(7):e159308). In this regard, a nanoporous membrane is one of the key components for a wearable and implantable bio-artificial kidney.

Lithium (Li) is the key material in Li-based devices and components, e.g, batteries, capacitors. The primary reserves of Li are estimated at over 250 billion tons (Xin Zhang, Aiguo Han and Yongan Yang, Review on the production of high-purity lithium metal, Journal of Materials Chemistry A 2020, 8, 22455-22466), of which 230 billion tons are within oceans, and the remaining as ores or continental brines. Due to the growing popularity and widespread applications of Li-based devices, demand for Li has been increasing rapidly. Li recycling of electronic waste becomes a very important source of Li closely after mining and extraction from natural resources.

Here, a new method for producing an active nanoporous membrane and the resulting product are disclosed.

### SUMMARY

A nanoporous membrane is fabricated by printing nanopillars of removable material on a substrate, performing deposition of at least one overlayer, and removal of the nanopillars to produce the nanoporous membrane. The nanopillar material may comprise a removable material, in which the material is removable by one or more of a solvent, heat, chemical treatment, and physical treatment.

In one configuration, the nanopillars are printed or deposited, for example by using 3D printing, nano-imprint, dip-pen lithography, laser writing or another suitable printing technique. By way of example, the material is printed or deposited at an aspect ratio having a height-to-width ratio of 3 to 100.

### BRIEF DESCRIPTION OF THE DRAWINGS

The descriptions that follow is further understood when read with the appended drawings. There are shown in the drawings exemplary embodiments of the disclosed technology for illustration purpose. The invention is not limited to the specific methods, compositions, and devices disclosed. Further, the drawings are not necessarily drawn to scale or proportion.

FIG. 1 is a schematic drawing illustrating a basic fabrication process of the disclosed technique.

FIG. 2 is a scanning electron micrograph (SEM) image showing the photoresist nanopillars prepared by 3D printing.

FIG. 3 is a schematic drawing illustrating the fabrication process for nanoporous membrane with inner wall surfaces modified.

FIG. 4 is a schematic drawing illustrating a typical capacitive deionization device for metal ion recycling.

FIG. 5 is a schematic drawing illustrating the fabrication of a nanoporous membrane prepared by the disclosed technique for lithium recycling.

FIG. 6 is a schematic drawing illustrating a stack of electrode/separator/electrode nanoporous membranes for lithium recycling.

### DETAILED DESCRIPTION

### Overview

The disclosed technique was devised to overcome the problems and challenges concerning the fabrication of functional nanoporous membranes. The following detailed description with reference to the drawings illustrates the spirit and essence of the disclosed technique. The illustrative embodiments and examples in the description, drawing, and claims are not meant to be limiting. Other embodiments may be utilized, and other changes may be made, without departing from the spirit of the subject matter presented here.

The disclosed technology pertains to a nanoporous membrane fabrication method and related applications. An array of sacrificial nanopillars and/or nanostructures of other shape and morphology of removable materials are printed onto a substrate. After serial deposition of over layers of even dissimilar nature (organic, inorganic, and biomaterials), the sacrificial nanostructures are dissolved, leaving a nanoporous membrane with nanopores, channels and cavities of nanoscale dimension and geometry designed, which enables specific and unique functions in different technological areas such as biological artificial organs, nanoelectronics, bioelectronics, molecular sensors, biomedical applications, etc.

The disclosed technique implements a method for fabrication of nanoporous membrane, in which nanopillars of removable material are printed on a substrate, followed by deposition of overlayer or overlayers, and subsequently, the removal of nanopillars to produce the nanoporous membrane. The substrate can be made of metal, non-metal, organics, inorganics and biomaterials, or combination of these materials.

In one configuration, the material printed as nanopillars is a removable material. The nanopillar layer may be removed by solvent, by heat, chemical treatment, physical treatment, or a combination of these techniques. The overlayer deposited onto the substrate with nanopillars can be metal, non-metal, organic, inorganic and biomaterial, or a combination of these materials. The overlayer can be deposited by spincasting, chemical deposition and physical deposition.

The nanopillars can be printed or deposited using 3D printing, nano-imprint, dip-pen lithography, laser writing and any printing technique. The printing technique used can be any technique capable of printing a high-aspect ratio nanopillar, and may have high-aspect ratio ranges from a height-to-width ratio of 3 to 100.

In another configuration, the nanopillar may have a preferred diameter from 1 nm to 1000 nm, and may have a preferred height from 100 nm to 10,000 nm. The nanopillars may be provided with a cylindrical shape or conical shape.

The technique may be used for preparing a surface functionalizing an inner wall of nanocavities of the nanoporous membrane, by preparing the nanopillars on a substrate, and exposing the nanopillars on the substrate to a solution containing the self-assemble molecules. The self-assemble molecules in this technique may have one end attached with a chemical functional moiety bestowing the desired surface property that can self-assemble onto the nanopillar surface. The other end is attached with a chemical functional moiety that can self-assemble onto the overlayer surface exposed during removal of nanopillars. The overlayer or overlayers may then be deposited, and nanopillars are removed, to produce the nanoporous membrane with a functionalized inner wall surface.

One end of the self-assemble molecules is attached with a chemical moiety bestowing the desired surface property, and can bind onto the nanopillar surface via weak chemical and/or physical interactions such as but not limited to electrostatic interaction, ionic bonds, weak chemical bonds; these interactions between the self-assemble molecules and the surface of nanopillars can be broken by chemical and/or physical treatment. The ending chemical moiety and/or its adjacent chemical functional groups that attach onto the surface of the nanopillar possesses a desired surface and chemical property of the final inner wall of a pore. The other end of the self-assemble molecules left hanging on the surface of the nanopillars that possesses another chemical functional moiety can bind strongly with the material of the overlayer to be deposited.

In another configuration, surface functionalizing of an inner wall of nanocavities of the nanoporous membrane is performed by preparing the nanopillars on a substrate, in which the precursor solution or material to be printed or deposited as nanopillars on the substrate, is mixed with self-assemble molecules before being printed or deposited onto the substrate surface. The self-assemble molecules have one end attached with a chemical functional moiety that can strongly bind with the overlayer material to be deposited and can attach firmly onto the overlayer material on the inner surface of the pore during removal of nanopillars to produce the nanoporous membrane with the functionalized surface on the inner wall. The other end of the self-assemble molecule is attached with a chemical functional moiety bestowing the desired surface property and is left hanging on the inner wall surface of the pore after removal of nanopillars to produce the nanoporous membrane. The overlayer or overlayers are then deposited and nanopillars are removed to produce the nanoporous membrane with the functionalized surface on the inner wall.

The nanoporous membrane may be used to fabricate a wearable and implantable bioartificial kidney, in which the bioartificial kidney has a multi-layered structure consisting of single or multiple functional overlayers.

The nanoporous membrane may be used to provide one or more layers functionalized for different purposes such as, but not limited to, drug delivering, antibacterial, antimicrobial, or electrically conducting functions.

In another configuration, the nanoporous membrane maybe used for lithium recovery, using a multi-layered structure consisting of single or multiple functional overlayers. One or more layers are functionalized and/or fabricated for lithium intercalation, lithium adsorption, lithium absorption, lithiation, and/or delithiation. One or more layers are functionalized and/or fabricated as electrodes through which positive or negative potential can be applied. One or more layers are functionalized and/or fabricated for intercalation, adsorption or absorption of other metals or metal ions.

### Basic Fabrication Process

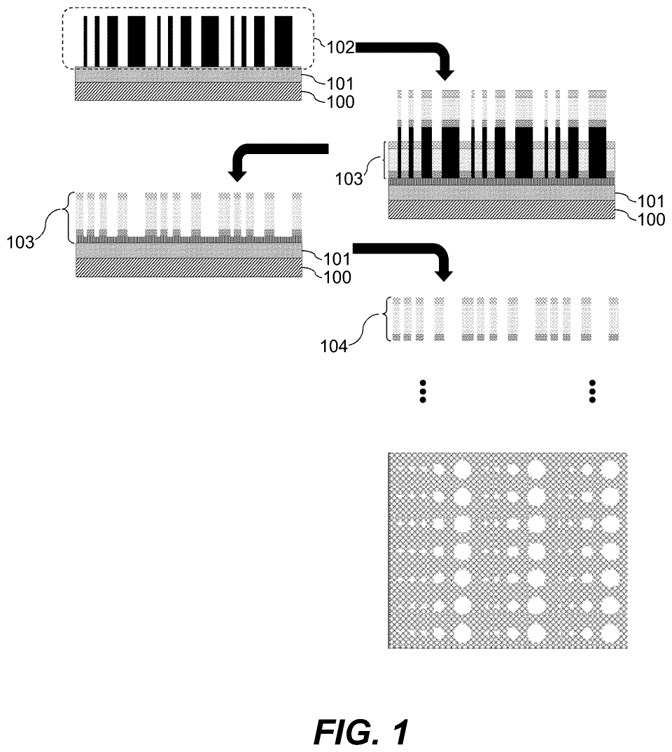

FIG. 1 is a schematic drawing illustrating a basic fabrication process of the disclosed technique. On a supporting substrate 100, a removable or dissolvable layer 101 is coated. On top of it, nanoscale cylindrical or conical pillars 102 can be 3D-printed or fabricated using lithographic techniques, leaving gaps and cavities in between. Compared with lithographic techniques, 3D-printing may have advantages in the disclosed technique because of its ease and lower cost of operation. It also offers a higher degree of freedom and flexibility in designing the nanoscale features. Advantageously, 3D-printing achieves high-aspect ratio nanoscale structures, which is difficult with conventional lithographic techniques. It is noted that this does not mean that the method of preparing the removable layer 101 is limited to 3D-printing. The choice of preparation method does not affect the legitimacy of the disclosed technique.

The substrate 100 serves primarily as a support. It can be a metal, ceramic, polymer or any material with enough mechanical strength and rigidity to support the nanostructures to be made above and the corresponding fabrications, and with enough chemical inertness towards the chemicals involved in subsequent processes. Although it serves as a support, the substrate 100 can be made of solid or porous materials. For the latter, it can be porous in nanoscale and serve as a filter itself.

Advantageously, layer 101 may be made of dissolvable material that can removed by solvent, by heat, or other appropriate treatment. For example, it can be poly methyl methacrylate (PMMA) that can be removed by acetone. For some material layer 101, heat or UV curing may be required to obtain nanopillars strong enough to survive subsequent processes. This layer 101 is optional. It mainly serves as a sacrificial interlayer for easy detachment of the final nanoporous membrane.

Afterwards, overlayers of materials can be deposited one by one as a stack of multi-layers 103. The stack can include overlayers of different material nature with different characteristics or functions if they do not react with each other. As such, thin film devices can be constructed around the nanopillars printed or deposited.

The next step is the removal of layer 101 and 102. Depending on the material properties, it would be dissolved by solvent, by wet chemical etching, by heat, or other appropriate treatment. This leaves the material from multilayers 103. Multilayers 103 are layers with nanopores whose size and morphology are dictated by those of nanopillars 104.

After this final treatment, the dimension of the 3D-printed nanopillar would dictate the dimension and morphology of the nanocavity to be constructed. For example, if 100 nm wide circular nanopillars are 3D-printed, cylindrical nanochannels with a diameter of around 100 nm are formed in the nanoporous membrane. As such, there is extremely high manufacturing flexibility and control of the dimension of the nanocavity required in the nanoporous membrane. Hitherto, an advanced commercial 3D printer can already achieve a linewidth of 50 nm (https://phenix81.com/plus/view.php?aid=26).

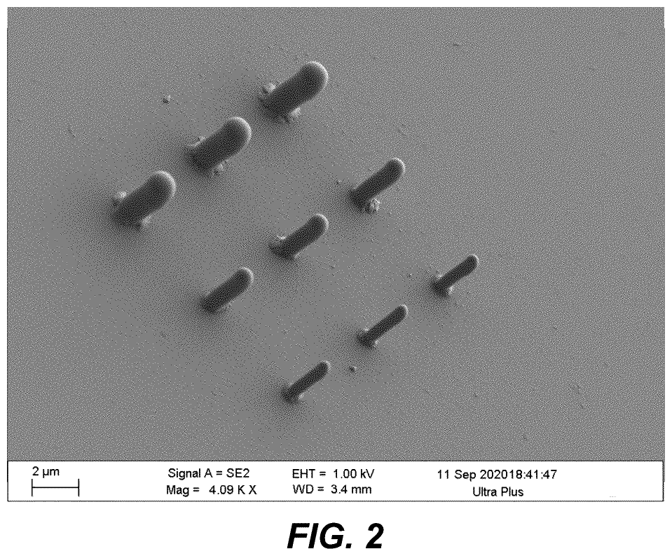

FIG. 2 is a scanning electron micrograph (SEM) image showing photoresist nanopillars prepared by 3D printing. The SEM shows an example of a 3×3 array of photoresist nanopillars 3D printed on a silicon substrate. The array has three rows of nanopillars of three diameters: 1125 nm, 750 nm, and 500 nm. All of them are 2 μm tall.

On the other hand, these nanocavities created will be surrounded by the stacked overlayers left behind, which can be pre-designed to serve as an active component, e.g., electrodes, antennas, heat circuit, etc.

### Surface Modification

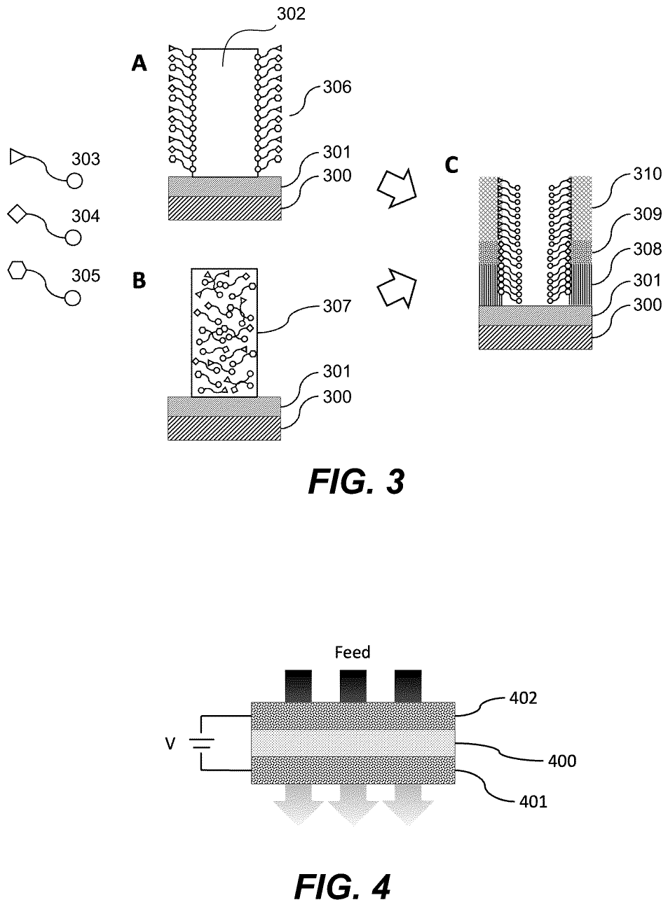

Besides bare nanocavities, the disclosed technique also discloses two methods to surface modify the inner walls of these nanocavities. Such a structure is useful, as one can modify the surface property of the inner walls of the nanocavities. FIG. 3 is a schematic drawing illustrating the fabrication process for a nanoporous membrane with inner wall surfaces modified.

Layers 300 and 301 are equivalent to 100 and 101 in FIG. 1. Molecules 303, 304 and 305 are self-assemble molecules with a chemical functional moiety (denoted by circle) that can confer a particular surface property onto a surface, e.g., hydrophilic, hydrophobic, charge bearing, etc. At the other end of the molecule is a chemical functional moiety (denoted by triangle, square and hexagon), which specifically react with a particular layer within the stack of overlayers. Although three overlayers are shown in the illustration (308, 309, 310), there is no limitation to the number of overlayers, so long as their combined thickness does not exceed the height of the printed nanopillars.

### Method A

The first disclosed method is to first achieve the printed nanopillars on a substrate as aforementioned, in which 300, 301 and 302 are equivalent to 100, 101 and 102 in FIG. 1. Still referring to FIG. 3, these nanopillars are exposed to a solution containing the self-assemble molecules 303, 304 and 305, which self-assemble onto the surface of nanopillar 302 randomly as stack 306, shown in FIG. 3 at A. The particular arrangement of stack 306 is given as a non-limiting example for illustrative purposes, as the order of the self-assemble can vary or can be random. In this particular method, it is required that chemical functional moiety bestowing the desired surface property (circle in FIG. 3) will self-assemble onto the nanopillar surface. The functional moieties at the other ends (triangle, square, hexagon in FIG. 3) will self-assemble onto a specific overlayer exposed within the nanocavities through with which this particular self-assemble molecule specifically interact with. The binding between a self-assemble molecule can be through electrostatic interaction, chemical- or bio-conjugation. It finally results in an inner nanocavity wall modified by the chemical functional moiety offering the desired surface property (denoted by circle in FIG. 3).

### Method B

Still referring to FIG. 3, the second disclosed method to surface modify the inner wall of nanocavities is restricted to the nanopillars printed which can be removed by solvent dissolution or other wet chemical methods. Firstly, appropriate self-assemble molecules 303, 304 and 305 are mixed with the “ink” to be printed onto the substrate, forming nanopillars made up of this mixture 307. Nanopillars are then printed onto the substrate, shown in FIG. 3 at B. Upon removal of the printed nanopillars upon solvent dissolution or other wet chemical methods, these self-assemble molecules are released. Each of these self-assemble molecules can then be free to attach to the surface of a specific overlayer exposed within the nanocavities, with which this self-assemble molecule specifically interacts with. The binding between a self-assemble molecule can be through electrostatic interaction, chemical- or bio-conjugation. It finally results in an inner nanocavity wall modified by the chemical functional moiety offering the desired surface property (denoted by circle in FIG. 3).

If an appropriate self-assemble molecule is chosen, selective modification on an inner wall of the nanocavity can be achieved as illustrated in FIG. 3 at C.

### Example 1: Membrane for Wearable and Implantable Bioartificial Kidney

An Si nanoporous membrane is mostly explored for this purpose, which enables glomerulus filtration. Durability and clotting of the blood filter in such bio-artificial kidneys are key concerns, in which the material characteristic (e.g., biocompatibility, flexibility, anticoagulant activity, etc.) and configuration (e.g., pore size and distribution) of the nanoporous membrane involved play a pivotal role. The present disclosure offers a versatile and flexible method to prepare such a nanoporous membrane.

Based on the present disclosure, desirable nanopores or nanochannels can be firstly constructed by 3D printing nanopillars of the desirable size and geometry, to effectively filter harmful substances from blood. By use of the disclosed technique, as the size and geometry of nanopores or nanochannels fabricated can be finely controlled, and selective filtration of a single entity or multiple entities are straightforward.

A biocompatible polymer can be deposited or spincast onto the substrate with nanopillars, which possesses the targeted physicochemical properties, such as wettability, stiffness, chemical inertness, etc. Furthermore, if a certain surface property is required, the nanocavities to be generated can be surface modified, for example by using Method A or Method B described above, with suitable chemical agents for better hemocompatibility, in the aspects of surface coagulation, protein adhesion, etc. Finally, a free-standing nanoporous membrane with uniform and well-controlled pore size and surface properties can be prepared after removing the printed nanopillars.

### Example 2: Nanoporous Membrane for Lithium Recovery

Capacitive deionization (CDI) is one of the methods to recycle Li. FIG. 4 is a schematic drawing illustrating a typical capacitive deionization device for metal ion recycling. One type of CDI device is that composed of a porous separator 400 sandwiched by a pair of porous membrane electrodes 401 and 402 as depicted in FIG. 4. The separator 400 serves to facilitate the fluid flow and to electrically isolate the two electrodes 401 and 402. When a certain voltage is applied to the electrodes, the Li recovery from this electro-adsorption process was found more efficient and selective from a multi-component aqueous solution (Dong Hee Lee, Taegong Ryu, Junho Shin, Jae Chun Ryu, Kang-Sup Chung and Young Ho Kim, Selective lithium recovery from aqueous solution using a modified membrane capacitive deionization system 2017, 173, 283-288).

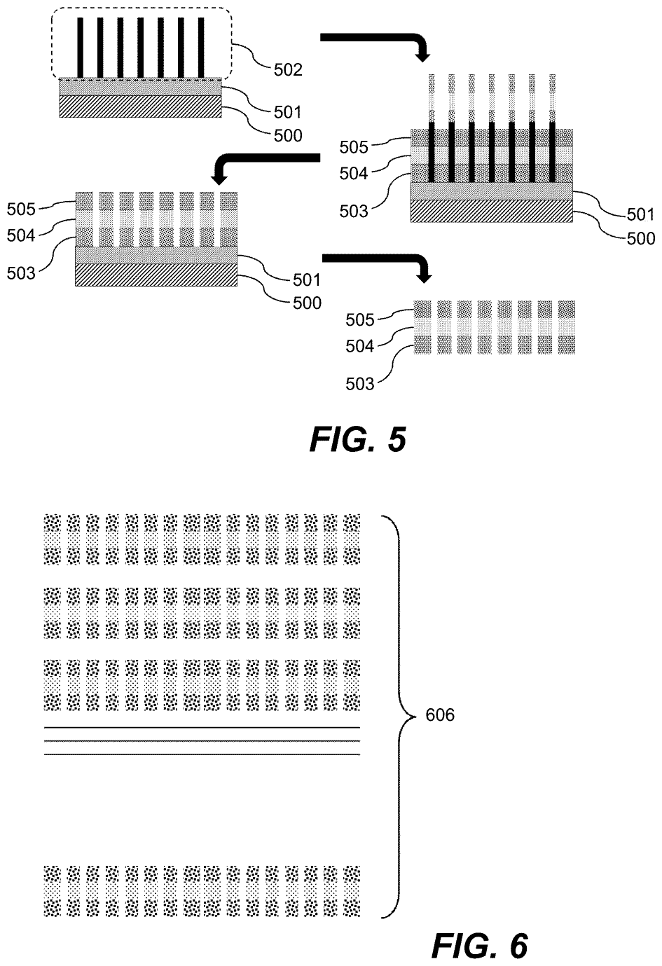

By using the disclosed techniques, the porous electrode/separator/electrode membrane can be prepared and used as a CDI device for Li recycling with highly controllable porosity and pore size. FIG. 5 is a schematic drawing illustrating the fabrication of a nanoporous membrane prepared by the disclosed technique for lithium recycling. First, nanopillars 502 of desirable dimension that will dictate the dimension of the nanocavities in the final product, are 3D-printed as described above, where 500 and 501 are equivalent to 100 and 101 in FIG. 1. Afterwards, a metal layer 503, an insulating layer 504 and another metal layer 505 are deposited sequentially as the bottom electrode, separator, and the top electrode to form a CDI device. To improve the Li ion selectivity and capacity, an additional layer or more layers of Li-selective material can be added within the layered structure, such as on top of the top electrode 505 or beneath the bottom electrode, which can store Li ions by Faradaic reaction, pseudo-capacitance, intercalation or insertion into the bulk. Manganese oxide (MnO₂) and iron phosphate (FePO₄) are two examples of these Li-selective materials (M. Pasta, C. D. Wessells, Y. Cui, F. La Manita, “A desalination battery”, Nano Letters 2012, 12, 839-843). Finally, all nanopillars 502 and layer 501 are removed, leaving a free-standing nanoporous membrane with a unique sandwich structure of an electrode/separator/electrode.

FIG. 6 is a schematic drawing illustrating a stack of electrode/separator/electrode nanoporous membrane for lithium recycling. The sandwich membranes can be packed into a stack 606 for treatment of high volume of waste feed for Li recycling. The porosity and pore size can also be adjusted to optimize performance and selectivity.

### CLOSING STATEMENT

The descriptions and examples herein are intended as non-limiting examples to serve to demonstrate the disclosed technology, and can be modified by one having ordinary skill in the art to which the claimed invention pertains within the scope of the subject matter of the claimed invention. On the other hand, the present invention is not limited by the examples disclosed in the specification of the subject application, and the scope of the present invention should be interpreted based on the claims, and to include all techniques that are within the equivalent scope.

From the foregoing, it will be appreciated that various embodiments of the present disclosure have been described herein for purposes of illustration. The present invention is not limited necessarily to the embodiments specifically disclosed, but that substitutions, modifications, and variations may be made to the present invention and its uses without departing from the spirit and scope of the invention. Accordingly, the various embodiments disclosed herein are not intended to be limiting, with the true scope and spirit being indicated by the following claims.

What is claimed is:

1. A method for fabrication of nanoporous membrane, comprising:
printing nanopillars of removable material on a substrate;
performing deposition of at least one overlayer; and
removal of the nanopillars to produce the nanoporous membrane.

2. The method of claim 1, wherein the material printed as nanopillars comprises a removable material, said removable material removable by one or more of a solvent, heat, chemical treatment, and physical treatment.

3. The method of claim 1, further comprising:
printing or depositing the nanopillars using 3D printing, nano-imprint, dip-pen lithography, laser writing and any printing technique, able to print at an aspect ratio having a height-to-width ratio of 3 to 100.

4. The method of claim 1, wherein the nanopillar has a diameter from 1 nm to 1000 nm, and a height from 100 nm to 10,000 nm.

5. The method of claim 1, wherein the nanopillars have at least one of a cylindrical shape, conical shape and spherical shape.

6. The method of claim 1, further comprising:
depositing the overlayer by one of spincasting, chemical deposition and physical deposition.

7. The method of claim 1, wherein the substrate comprises a metal, or a combination of metal and another material.

8. The method of claim 1, wherein the substrate comprises a non-metal, or a combination of a non-metal and another material.

9. The method of claim 1, wherein the substrate comprises a non-organic material and another material.

10. The method of claim 1, wherein the overlayer deposited onto the substrate comprises a metal, or a combination of metal and another material.

11. The method of claim 1, wherein the overlayer deposited onto the substrate comprises a non-metal, or a combination of a non-metal and another material.

12. The method of claim 1, wherein the overlayer deposited onto the substrate comprises an organic material or an organic material and another material.

13. The method of claim 1, wherein the overlayer deposited onto the substrate comprises a non-organic material or a non-organic material and another material.

14. A method of surface functionalizing inner wall of nanocavities of the nanoporous membrane, comprising:
preparing the nanopillars on a substrate by fabrication of a nanoporous membrane as described in claim 1; and
prior to performing the deposition of said at least one overlayer, exposing the nanopillars on substrate to a solution comprising self-assemble molecules, the self-assemble molecules having one end attached with a chemical functional moiety bestowing the desired surface property to self-assemble onto the nanopillar surface; and the other end attached with a chemical functional moiety to self-assemble onto the overlayer surface exposed during removal of nanopillars,
wherein the removal of nanopillars produces the nanoporous membrane with an inner nanocavity wall modified by the chemical functional moiety.

15. The method of claim 14, wherein the one end of the self-assemble molecules is attached with a chemical moiety bestowing a surface property to bind onto the nanopillar surface via chemical and/or physical interactions between the self-assemble molecules and the surface of nanopillars be broken by chemical and/or physical treatment.

16. A method of surface functionalizing inner wall of nanocavities of the nanoporous membrane, comprising:
preparing the nanopillars on a substrate by fabrication of a nanoporous membrane as described in claim 1, using a precursor solution or material to be printed or deposited as nanopillars on the substrate mixed with self-assemble molecules before being printed or deposited onto the substrate surface, the self-assemble molecules having one end attached with a chemical functional moiety to bind with the overlayer material to be deposited and to attach onto the overlayer material on the inner surface of the pore during removal of nanopillars to produce the nanoporous membrane with functionalized surface on the inner wall, and the other end of the self-assemble molecule attached with a chemical functional moiety bestowing a predetermined surface property on an inner wall surface of a pore after removal of nanopillars to produce the nanoporous membrane; and
performing said removal of nanopillars to produce the nanoporous membrane with functionalized surface on the inner wall.

17. A nanoporous membrane for wearable and implantable bioartificial kidney, comprising:
a multi-layered structure consisting of single or multiple functional overlayers fabricated using the method of claim 1.

18. A nanoporous membrane for lithium recovery, comprising:
a multi-layered structure consisting of single or multiple functional overlayers fabricated using the method of claim 1.

19. The nanoporous membrane of claim 18, wherein one or more layers provide at least one of the group of lithium interactions selected from lithium intercalation, lithium adsorption, lithium absorption, lithiation, and delithiation.

20. The design of claim 18, wherein one or more layers function as electrodes through which positive or negative potential can be applied.

21. The design of claim 18, wherein one or more layers function to provide intercalation, adsorption or absorption of other metal or metal ions.
